(19) United States
(12) Patent Application Publication
(10) Pub. No.: US 2022/0039492 A1
(43) Pub. Date: Feb. 10, 2022

(54) REUSABLE SMART MEDICAL PROTECTIVE MASK

(71) Applicant: Hong Kong Green Dreamer Technology Limited, Hong Kong (CN)

(72) Inventor: KAM-MEI MA, HONG KONG (CN)

(21) Appl. No.: 17/393,792

(22) Filed: Aug. 4, 2021

(30) Foreign Application Priority Data
Aug. 4, 2020 (CN) 20201598040.8

(51) Int. Cl. A41D 13/11; A61L 9/20; A61L 9/22; A41D 13/002; A41D 13/00

(52) U.S. Cl. CPC A41D 13/1153; A61L 9/20; A61L 9/22; A61L 2209/14; A41D 13/0002; A41D 13/1184; A41D 13/002

(57) ABSTRACT

A reusable smart medical protective mask is provided, including a mask body, a body temperature monitoring system, a breathing filter system and a photoelectric function system. The mask body includes a head accommodating cavity, a face mask part, a first air inlet channel and an air outlet channel. The body temperature monitoring system includes a temperature sensor disposed in the head accommodating cavity, a controller and a warning device disposed on the outer surface of the mask body. The breathing filter system includes a first air supply device disposed in the first air inlet channel and a second air supply device disposed in the air outlet channel. The photoelectric function system includes a photoelectric conversion device fixed on the outer surface of the mask body and an energy storage device electrically connected to the body temperature monitoring system and the breathing filter system.

### CROSS-REFERENCE TO RELATED APPLICATION

This application claims the benefit of Chinese Application No. 20201598040.8, filed on Aug. 4, 2020, the disclosure of which is incorporated herein in its entirety by reference.

### BACKGROUND OF THE INVENTION

### 1 Field of the Invention

The present invention generally relates to the technical field of medical devices, in particular to a reusable smart medical protective mask.

### 2. Description of the Related Art

A part of the diseases are spread by droplets, and protective masks are the effective protective equipment to deal with these diseases. The conventional protective masks are usually full cover types and breathing through the screening program to ensure the protection effect. However, due to the obstruction of the screening program, the medical staff is particularly strenuous in breathing. The difficulty of breathing is increased and the wearing experience is not good enough.

In addition, for the medical personnel, they have more contact with the patients when helping the patients and may be infected at any time. If the medical personnel are infected, it may further infect to more patients. Therefore, the medical personnel need to be monitored, but the conventional masks do not have such functions.

Therefore, a new technology is needed to solve the problems that the wearing experience of the protective masks in the conventional technology is not good enough and the medical personnel cannot be monitored.

### SUMMARY OF THE INVENTION

To solve the foregoing problems in the conventional technology, the present disclosure provides a reusable smart medical protective mask, which disposes an air supply device to assist breathing, so as to guarantee a comfortable breathing and to set up a temperature sensor for monitoring the user's body temperature in real time.

The present disclosure adopts the following solutions:

A reusable smart medical protective mask is disclosed. The reusable smart medical protective mask includes a mask body, a body temperature monitoring system, a breathing filter system and a photoelectric function system. A head accommodating cavity is disposed inside the mask body. A face mask part is disposed at the front side of the mask body and the face mask part is transparent. The mask body further includes a first air inlet channel and an air outlet channel, the first air inlet channel and the air outlet channel are connected to the head accommodating cavity. The body temperature monitoring system includes a temperature sensor, a controller and a warning device. The temperature sensor and the warning device are electrically connected to the controller. The temperature sensor is disposed in the head accommodating cavity. The warning device is disposed on outer surface of the mask body. The breathing filter system includes a first air supply device disposed in the first air inlet channel and a second air supply device disposed in the air outlet channel. The first air inlet channel and the air outlet channel have a screening program. The first air supply device and the second air supply device are electrically connected to the controller. The photoelectric function system includes a photoelectric conversion device and an energy storage device electrically connected to the photoelectric conversion device. The energy storage device is electrically connected to the body temperature monitoring system and the breathing filter system. The photoelectric conversion device is fixed on the outer surface of upper end of the mask body.

As a further improvement of the technical solution in the present disclosure, a soft pad is disposed on inner wall of the head accommodating cavity and the temperature sensor is fixed on the pad and disposed near a forehead area.

As a further improvement of the technical solution in the present disclosure, two sides of the face mask part are rotatably connected with the mask body. A rotation driving device is disposed in the mask body for driving the face mask part to rotate corresponding to the mask body. The rotation driving device is electrically connected to the controller and the energy storage device.

As a further improvement of the technical solution in the present disclosure, the reusable smart medical protective mask further includes a smart glasses disposed up and down on inner side of the face mask part. The smart glasses includes a glasses frame, an adjustable lens fixed on the glasses frame for displaying image and a control command input part disposed in the glasses frame. Two sides of the glasses frame are pivotally connected with the mask body to flip up and down. The adjustable lens is electrically connected to the controller and the control command input part is electrically connected to the controller.

As a further improvement of the technical solution in the present disclosure, the reusable smart medical protective mask further includes a negative ion generation device. The mask body further includes a second air inlet channel connected to the head accommodating cavity. The breathing filter system further includes a third air supply device disposed in the second air inlet channel and the second air inlet channel has the screening program. The negative ion generation device is disposed in the second air inlet channel and is located behind the third air supply device. The negative ion generation device and the third air supply device are electrically connected to the controller and the energy storage device.

As a further improvement of the technical solution in the present disclosure, the reusable smart medical protective mask further includes a first sound collection device disposed in the head accommodating cavity and located under the face mask part, a first speaker disposed in the head accommodating cavity, a second sound collection device disposed on the outer surface of the mask body, and a second speaker disposed on the outer surface of the mask body. The first sound collection device, the second sound collection device and the second speaker are electrically connected to the controller and the energy storage device.

As a further improvement of the technical solution in the present disclosure, the reusable smart medical protective mask further includes a wireless communicator for wireless communication with an external apparatus. The wireless communicator is electrically connected to the controller and the energy storage device.

As a further improvement of the technical solution in the present disclosure, the first air supply device and/or the second air supply device is a mini air extraction fan, and a nano photocatalyst coating is disposed on blades of the mini air extraction fan.

As a further improvement of the technical solution in the present disclosure, both sides the first air inlet channel and/or the second air inlet channel include a silicone nano fiber protective film and the silicone nano fiber protective film is electrostatically treated.

As a further improvement of the technical solution in the present disclosure, the reusable smart medical protective mask further includes an ultraviolet germicidal lamp disposed in the head accommodating cavity. The ultraviolet germicidal lamp is electrically connected to the controller and the energy storage device.

Compared with the conventional technology, the beneficial effects of the present invention are as follows. In the reusable smart medical protective mask of the present disclosure, the body temperature monitoring system and the breathing filter system are provided. The body temperature monitoring system may monitor the body temperature of the user in real time and send it to the controller. If the body temperature is too high, the shell body may warn through the warning device. In the breathing filter system, the first air supply device and the second air supply device are respectively disposed in the first air inlet channel and the sir outlet channel, and the first air inlet channel and the air outlet channel respectively have a screening program. The air circulation in the protective mask can be ensured by the air supply from the first air supply device and the second air supply device, so as to guarantee the comfortable breathing for the user. In addition, the photoelectric function system is disposed. The photoelectric conversion device of the photoelectric function system may convert the overhead lights or the outdoor sunlight and store it in the energy storage device for supplying power to other electrical components, so as to save the electric energy.

### BRIEF DESCRIPTION OF THE DRAWINGS

The technology of the present disclosure will be described in more details hereinafter with reference to the accompanying drawings and the specific embodiments as follows.

FIG. 1 is a schematic diagram of the reusable smart medical protective mask in accordance with the present disclosure.

FIG. 2 is a schematic diagram of the reusable smart medical protective mask in accordance with the present disclosure when the front mask body and the back mask body are opened.

FIG. 3 is a front view diagram of the mask body of the reusable smart medical protective mask in accordance with the present disclosure.

FIG. 4 is a cross-sectional diagram of the first air inlet channel and the air outlet channel in accordance with the present disclosure.

FIG. 5 is a cross-sectional diagram of the second air inlet channel in accordance with the present disclosure.

FIG. 6 is a schematic diagram of the electrical connection of the electrical components in accordance with the present disclosure.

### DETAILED DESCRIPTION OF THE PREFERRED EMBODIMENTS

Hereinafter, the conception, the specific structure and the technical effects of the present disclosure will be clearly and fully described with reference to the embodiments and the accompanying drawings so that the objects, the aspects and the effects of the present disclosure can be fully understood. It is noted that the embodiments in the present disclosure and the technical features in the embodiments can be combined with each other if there is no conflict. Similar reference numerals in the accompanying drawings designate the same or the similar elements throughout the specification.

It is noted that, if there is no specific description, when a feature is called “fixed” or “connected” to another feature, it can be directly fixed or connected to another feature, or indirectly fixed or connected to another feature. Besides, the terms “up, down, left, right” used in the present disclosure only describe the positional relation between each component in accordance with the accompanying drawings of the present disclosure.

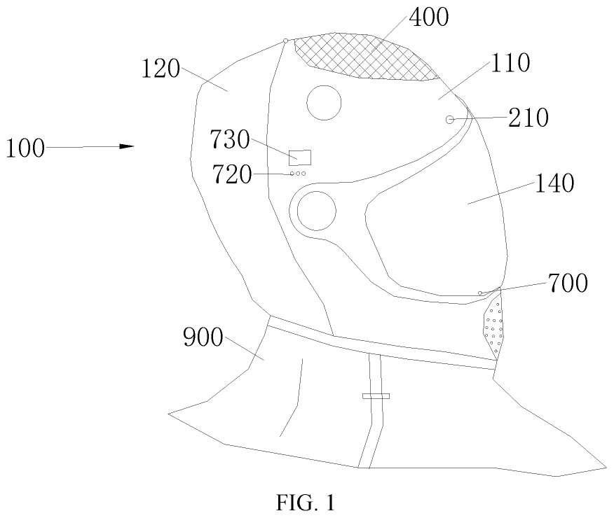

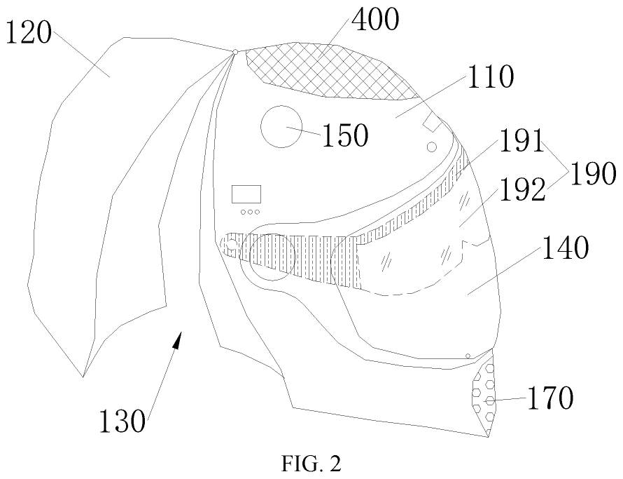

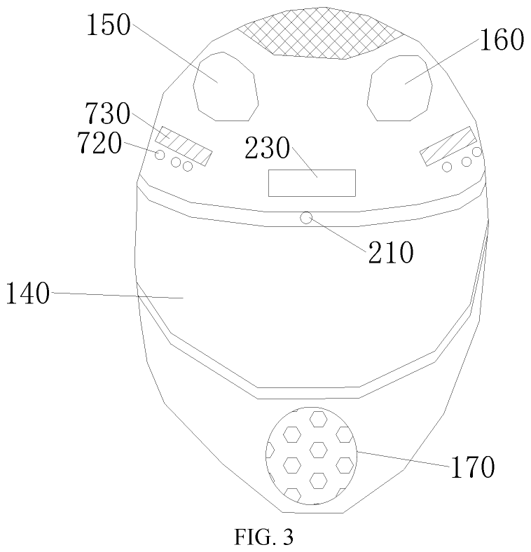

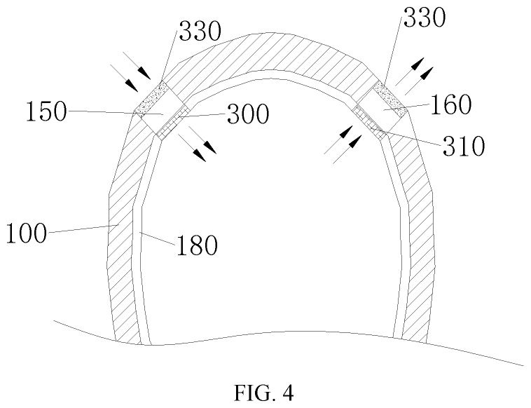

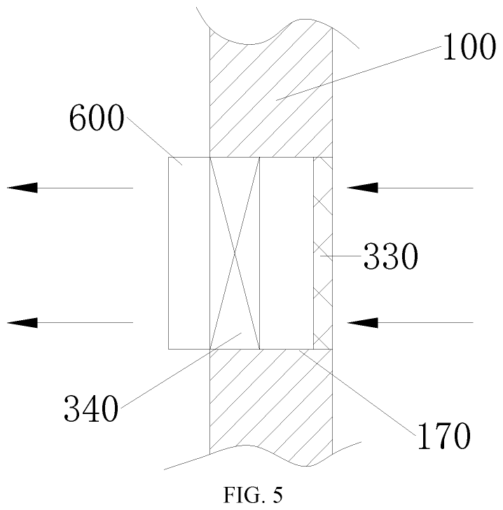

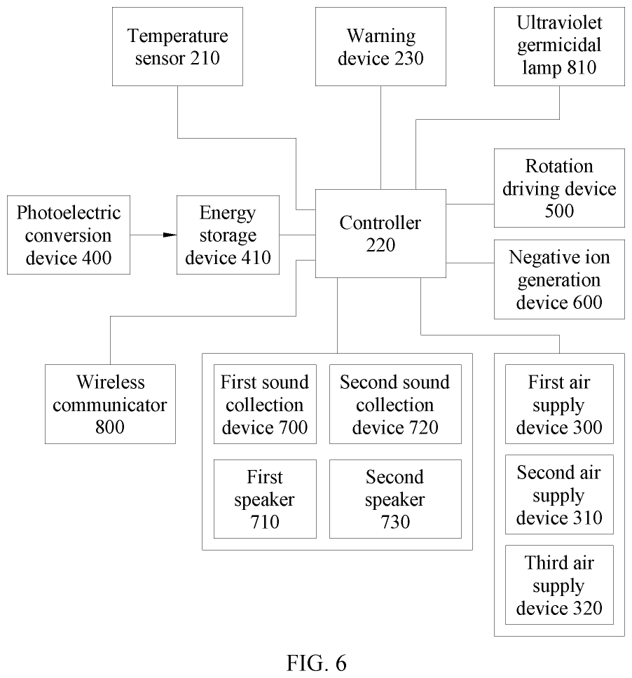

Please refer to FIG. 1 to FIG. 6, the reusable smart medical protective mask of the present disclosure includes a mask body 100, a body temperature monitoring system, a breathing filter system and a photoelectric function system.

The mask body 100 includes a front mask body 110 and a back mask body 120, which can be in open connection or in close connection. When the front mask body 110 and the back mask body 120 are closed, a head accommodating cavity 130 is formed inside. The entire head of the person can be covered, and a reserved space for the neck is formed under the head accommodating cavity 130 when in the close connection. A transparent face mask part 140 is disposed at the front side of the mask body 100 (that is, on the mask body 100), so that the user may view the outside world through the face mask part 140. At the same time, the face mask part 140 isolates the human face from the outside world to avoid the infection. The mask body 100 further includes a first air inlet channel 150 and an air outlet channel 160. The first air inlet channel 150 and the air outlet channel 160 are connected to the head accommodating cavity 130, so as to exchange air easily from the outside world. Wherein, the first air inlet channel 150 and the air outlet channel 160 are respectively disposed on left and right sides of the mask body 100.

The body temperature monitoring system includes a temperature sensor 210, a controller 220 and a warning device 230. The temperature sensor 210 and the warning device are electrically connected to the controller 220. The temperature sensor 210 is disposed in the head accommodating cavity 130. The warning device 230 is disposed on outer surface of the mask body 100. The temperature sensor 210 may monitor the body temperature of the user and the information of the body temperature is sent to the controller 220. If the body temperature is higher than the preset value in the controller 220, the controller 220 may control the warning device 230 to remind the user. Specifically, the warning device 230 may be a warning light or a display screen.

The breathing filter system includes a first air supply device 300 disposed in the first air inlet channel 150 and a second air supply device 310 disposed in the air outlet channel 160. The first air inlet channel 150 and the air outlet channel 160 have a screening program 330. The first air supply device 300 and the second air supply device 310 are electrically connected to the controller 220. The first air supply device 300 sends the air from outside to inside and the second air supply device 310 extract the air from inside to outside, so that the air in the head accommodating cavity 130 can flow with the outside world. At the same time, the screening program 330 filters the air during the air supply process and the air extraction process, so as to avoid the external viruses entering the head accommodating cavity 130 or the virus in the head accommodating cavity 130 being discharged to the outside environment to cause the pollution. The first air supply device 300 and the second air supply device 310 are electrically connected to the controller 220 and controlled by the controller 220. When the mask is not worn on the head, the air supply and the air extraction are not processed. The task only activates when the mask is worn on the head.

Specifically, the first air supply device 300 and/or the second air supply device 310 is a mini air extraction fan, and a nano photocatalyst coating is disposed on blades of the mini air extraction fan. The nano photocatalyst coating plays a role of sterilization. When conducting the air supply process and the air extraction process, the air passing through the nano photocatalyst coating can be sterilized and the risk of infection can be further reduced.

The photoelectric function system includes a photoelectric conversion device 400 and an energy storage device 410 electrically connected to the photoelectric conversion device 400. The energy storage device 410 is electrically connected to the body temperature monitoring system and the breathing filter system. The photoelectric conversion device 400 is fixed on the outer surface of upper end of the mask body 100. The photoelectric conversion device 400 may be a solar panel, which is able to convert the light energy to the electric energy and to store it in the energy storage device 410 for saving the electric energy. The energy storage device 410 may be a battery. The technology of converting light energy into electric energy is a mature technology, and the related contents can refer to the conventional technology. Such contents will not go into details here.

Based on the above structure, in the reusable smart medical protective mask of the present disclosure, the body temperature monitoring system and the breathing filter system are disposed. The temperature sensor 210 of the body temperature monitoring system may monitor the body temperature of the user in real time and send it to the controller 220. When the body temperature is too high, the shell body may remind user by the warning device 230. In the breathing filter system, the first air supply device 300 and the second air supply device 310 are respectively disposed in the first air inlet channel 150 and the air outlet channel 160. The screening program 330 is respectively disposed in the first air inlet channel 150 and the air outlet channel 160. The air flow in the protective mask may be guaranteed by the air supply from the first air supply device 300 and the second air supply device 310, so as to guarantee the comfortable breathing for the user. At the same time, the screening program 330 may guarantee the air in and out of the mask is clean. In addition, the photoelectric function system is disposed. The photoelectric conversion device 400 of the photoelectric function system may convert the overhead lights or the outdoor sunlight and store it in the energy storage device 410, so as to supply power to other electrical components and to save the electric energy.

In order to increase the user's comfort of wearing the mask body 100, a soft pad 180 is disposed on the inner wall of the head accommodating cavity 130 to ensure that the wearer's head is comfortable. The shape of the pad 180 matches the shape of the human head. The pad 180 may be made by the materials having flexibility and more comfortable, like a sponge pad or a silicone pad. The temperature sensor 210 is fixed on the pad 180 and disposed near the forehead area. The forehead temperature of the wearer is measured to ensure the accuracy of the temperature monitoring.

Two sides of the face mask part 140 are rotatably connected with the mask body 100. A rotation driving device 500 is disposed in the mask body 100 for driving the face mask part 140 to rotate corresponding to the mask body 100. The rotation driving device 500 is electrically connected to the controller 220 and the energy storage device 410. A window is disposed at the area of the front side of the mask body 100 corresponding to the human eye. The face mask part 140 is used to seal the window, so as to prevent the outside air from directly entering the head accommodating cavity 130 to contact the wearer. The rotation driving device 500 may be an electric machine. The face mask part 140 is transparent and can be made by transparent plastic, transparent acrylic or other materials. The face mask part 140 is rotatably connected with the mask body 100 by a pin shaft. The pin shaft and the electric machine are in transmission connection through a transmission mechanism. The transmission mechanism may be a gear. The rotation of the electric machine may make the face mask part 140 rotates relative to the mask body 100, so as to open or close the face mask part 140. When the wearer is in a safe environment (for example, during the safe rest time), the face mask part 140 is raised by the controller 220 to expose the window and directly connect with the outside air. When the wearer is in a dangerous environment (for example, there is a possibility of infection), the face mask part needs to be controlled for sealing the window.

In the preferred embodiment, the reusable smart medical protective mask of the present disclosure further includes a smart glasses 190 disposed up and down on inner side of the face mask part 140. The smart glasses 190 includes a glasses frame 191, an adjustable lens 192 fixed on the glasses frame 191 for displaying image and a control command input part disposed in the glasses frame. Two sides of the glasses frame 191 are pivotally connected with the mask body 100 to flip up and down. The pivot is connected to the electric machine, which is able to control the up and down flip and lift. The adjustable lens 192 is electrically connected to the controller 220 and the control command input part is electrically connected to the controller 220. The adjustable lens 192 is a projection screen set up with a projector, and image or video can be projected by the adjustable lens 192. The glasses frame 191 disposes an optical waveguide tube, a display, an input coupler and an output coupler above the adjustable lens 192. The image can be projected or stop projecting by the commands. The appropriate image can be projected according to the actual situation. For example, when conducting a surgery, the corresponding images can be projected for instructions and to assist the operation of the medical personnel. The control command input part may allow the user to select the operation according to the image. The control command input part includes a touch sensor, a light sensor, a pressure sensor, a deformation sensor and so on, which may be installed on both sides of the glasses frame 191. The touch operation from the user can be collected by such sensors for inputting the command. That is, the smart glasses 190 is disposed and can be used as an augmented reality (AR) glasses or a virtual reality (VR) glasses. The following microphones and headphones can be cooperated to realize the voice control function. The detail structure of the AR glasses or the VR glasses may refer to the conventional technology. Such contents will not go into details here.

In the preferred embodiment, in order to further sterilize and purify the air in the head accommodating cavity 130, the reusable smart medical protective mask of the present disclosure further includes a negative ion generation device 600. The mask body 100 further includes a second air inlet channel 170 connected to the head accommodating cavity 130. The breathing filter system further includes a third air supply device 340 disposed in the second air inlet channel 170 and the second air inlet channel 130 has the screening program 330. The negative ion generation device 600 is disposed in the second air inlet channel 170 and is located behind the third air supply device 340. The negative ion generation device 600 and the third air supply device 340 are electrically connected to the controller 220 and the energy storage device 410. The second air inlet channel 170 is similar to the first air inlet channel 150. The air is supplied from outside to inside by the third air supply device 340, screened by the screening program 330, further sterilized and purified through the ion generation device 600, and finally entered the head accommodating cavity 130 for the wearer to breath. The ion generation device 600 is a miniature ion generation device and the volume is smaller. The ion generation device 600 may refer to the conventional technology. Such contents will not go into details here. The third air supply device 340 is also a mini air extraction fan. A net cover is disposed on the outer side of the third air supply device 340.

Since the mask body 100 covers the full head during the normal working condition, the mask body 100 is sealed expect of the air inlet channel and air outlet channel used for air circulation. Therefore, the communication to the outside world is not normally provided through the voice. To solve this problem, the reusable smart medical protective mask of the present disclosure further includes a first sound collection device 700 disposed in the head accommodating cavity 130 and located under the face mask part 140, a first speaker 710 disposed in the head accommodating cavity 130, a second sound collection device 720 disposed on the outer surface of the mask body 100, and a second speaker 730 disposed on the outer surface of the mask body 100. The first sound collection device 700, the second sound collection device 720 and the second speaker 730 are electrically connected to the controller 220 and the energy storage device 410. The first sound collection device 700 collects the voice of the wearer and the sound is played by the second speaker 730. The second sound collection device 720 collects the voice from the outside world and the sound is played by the first speaker 710, so that the wearer can hear the sound. Based on the cooperation from the first sound collection device 700, the second sound collection device 720, the first speaker 710 and the second speaker 730, the wearer may normally communicate to the outside world through their voice. The first sound collection device 700 and/or the second sound collection device 720 may be a microphone, preferably a carbon microphone. The first speaker 710 may be in-ear headphones or bone conduction headphones.

In addition, based on the cooperation from the first speaker 710 and the controller 220, the face mask part can open and close by the voice control and the start and stop of the air supply devices can also be controlled. For example, when the user wears the protective mask as mentioned in the present disclosure, the face mask part 140 moves down and closes by the voice control, and the air supply and the air extraction are operated by activating the air filter system.

In the preferred embodiment, the reusable smart medical protective mask of the present disclosure further includes a wireless communicator 800 for wireless communication with an external apparatus. The wireless communicator 800 is electrically connected to the controller 220 and the energy storage device 410. The wireless communicator 800 may be a Bluetooth communicator and the external apparatus may be a smart phone. The measured body temperature is sent to the smart phone to display and to remind the user. The Bluetooth communicator can be used to answer the phone.

In the preferred embodiment, in order to further increase protection, the reusable smart medical protective mask of the present disclosure further includes a neck protector 900 detachably connected to the mask body 100. The top structure of the neck protector 900 is small and the bottom structure is big. The bottom side is able to cover the shoulders and the top side can wrap the neck. The neck protector 900 is soft and can fit people's neck and shoulders. The neck protector 900 is made by the silicone or other materials. The neck protector 900 and the mask body 100 are detachably connected by a buckle or a tape sticker. The neck protector 900 has a switchable opening, and the opening is fixed by the tape sticker.

In the preferred embodiment, both sides of the first air inlet channel 150 and/or the second air inlet channel 170 include a silicone nano fiber protective film and the silicone nano fiber protective film is electrostatically treated. The tiny particles in the air can be absorbed and fixed to further purify the air.

In the preferred embodiment, the reusable smart medical protective mask of the present disclosure further includes ultraviolet germicidal lamp 810 disposed in the head accommodating cavity 130. The ultraviolet germicidal lamp 810 is electrically connected to the controller 220 and the energy storage device 410. When the mask is not wearing, the head accommodating cavity 130 is sterilized by the ultraviolet germicidal lamp 810. Of course, the ultraviolet germicidal lamp 810 is turned off when the mask is worn, so as to prevent the damage to the skin.

The other contents of the reusable smart medical protective mask described in the present disclosure may refer the conventional technology. Such contents will not go into details here.

The above are only the preferred embodiments of the present disclosure, and do not limit the present disclosure in any form. Therefore, any modifications, equivalent changes and variations made to the above embodiments based on the techniques of the present disclosure without departing from the content of the technical solutions of the present disclosure still fall within the scope of the technical solution of the present disclosure.

What is claimed is:

1. A reusable smart medical protective mask, comprising:
a mask body comprising a front mask body and a back mask body connected to each other, the front mask body and the back mask body being assembled to form a head accommodating cavity inside, the front mask body having a face mask part and the face mask part being transparent, the mask body further comprising a first air inlet channel and an air outlet channel, the first air inlet channel and the air outlet channel being connected to the head accommodating cavity;
a body temperature monitoring system comprising a temperature sensor, a controller and a warning device, the temperature sensor and the warning device being electrically connected to the controller, the temperature sensor being disposed in the head accommodating cavity and the warning device being disposed on outer surface of the mask body;
a breathing filter system comprising a first air supply device disposed in the first air inlet channel and a second air supply device disposed in the air outlet channel, the first air inlet channel and the air outlet channel having a screening program, the first air supply device and the second air supply device being electrically connected to the controller; and
a photoelectric function system comprising a photoelectric conversion device and an energy storage device electrically connected to the photoelectric conversion device, the energy storage device being electrically connected to the body temperature monitoring system and the breathing filter system, the photoelectric conversion device being fixed on the outer surface of upper end of the mask body.

2. The reusable smart medical protective mask of claim 1, wherein a pad is disposed on inner wall of the head accommodating cavity and the temperature sensor is fixed on the pad and disposed near a forehead area.

3. The reusable smart medical protective mask of claim 1, wherein two sides of the face mask part are rotatably connected with the mask body, a rotation driving device is disposed in the mask body for driving the face mask part to rotate corresponding to the mask body, the rotation driving device is electrically connected to the controller and the energy storage device.

4. The reusable smart medical protective mask of claim 1, further comprising a smart glasses disposed up and down on inner side of the face mask part, wherein the smart glasses includes a glasses frame, an adjustable lens fixed on the glasses frame for displaying image and a control command input part disposed in the glasses frame, two sides of the glasses frame are pivotally connected with the mask body to flip up and down, the adjustable lens is electrically connected to the controller and the control command input part is electrically connected to the controller.

5. The reusable smart medical protective mask of claim 1, further comprising a first sound collection device disposed in the head accommodating cavity and located under the face mask part, a first speaker disposed in the head accommodating cavity, a second sound collection device disposed on the outer surface of the mask body, and a second speaker disposed on the outer surface of the mask body, wherein the first sound collection device, the second sound collection device and the second speaker are electrically connected to the controller and the energy storage device.

6. The reusable smart medical protective mask of claim 1, further comprising a wireless communicator for wireless communication with an external apparatus, wherein the wireless communicator is electrically connected to the controller and the energy storage device.

7. The reusable smart medical protective mask of claim 1, further comprising a negative ion generation device, the mask body further comprising a second air inlet channel connected to the head accommodating cavity, the breathing filter system further comprising a third air supply device disposed in the second air inlet channel and the second air inlet channel having the screening program, wherein the negative ion generation device is disposed in the second air inlet channel and is located behind the third air supply device, the negative ion generation device and the third air supply device are electrically connected to the controller and the energy storage device.

8. The reusable smart medical protective mask of claim 1, wherein the first air supply device and/or the second air supply device is a mini air extraction fan, and a nano photocatalyst coating is disposed on blades of the mini air extraction fan.

9. The reusable smart medical protective mask of claim 7, wherein both sides of the first air inlet channel and/or the second air inlet channel comprise a silicone nano fiber protective film and the silicone nano fiber protective film is electrostatically treated.

10. The reusable smart medical protective mask of claim 1, further comprising an ultraviolet germicidal lamp disposed in the head accommodating cavity, wherein the ultraviolet germicidal lamp is electrically connected to the controller and the energy storage device.
